(19) United States
(12) Patent Application Publication
(10) Pub. No.: US 2026/0014076 A1
(43) Pub. Date: Jan. 15, 2026

(54) SURFACE PEGYLATED SOLID LIPID NANOCARRIER DUALLY LOADED WITH ATAZANAVIR AND ELVITEGRAVIR FOR COMBINATION ANTIRETROVIRAL THERAPY

(71) Applicant: VIRGINIA COMMONWEALTH UNIVERSITY, Richmond, VA (US)

(72) Inventors: Laleh GOLSHAHI, Glen Allen, VA (US); Arya Ahmadi BAZARGANI, Henrico, VA (US)

(21) Appl. No.: 19/057,442

(22) Filed: Feb. 19, 2025

Related U.S. Application Data
(60) Provisional application No. 63/556,026, filed on Feb. 21, 2024.

(51) Int. Cl. A61K 9/1271; A61K 9/1277; A61K 9/51; A61K 31/4402; A61K 31/47

(52) U.S. Cl. CPC A61K 9/1271; A61K 9/1277; A61K 9/5123; A61K 9/5192; A61K 31/4402; A61K 31/47

(57) ABSTRACT

A preparation of PEGylated solid lipid core nanocarriers (SLN) is loaded with at least one drug for delivery to a subject. SLNs having a small size, neutral charge, and high drug encapsulation efficiency are formed. PEGylation improves SLN permeability without hindering cellular uptake, particularly permeability of nasal mucous for intranasal delivery. PEGylated SLNs are suitable for intranasal, inhaled, oral or injected drug delivery. Exemplary formulations include a combined antiretroviral therapy (cART) designed to cross the blood-brain barrier and treat neuroAIDS.

### CROSS-REFERENCE TO RELATED APPLICATIONS

This application claims the benefit of U.S. Provisional Application No. 63/556,026, filed Feb. 21, 2024, the complete contents of which is hereby incorporated by reference.

### BACKGROUND OF THE INVENTION

### Field of the Invention

The invention generally relates to nanocarriers for drug delivery. The invention further relates to methods for the use of nanocarriers for delivery of antiviral therapies.

### Background

The direct nose-to-brain (N-t-B) delivery route has recently become a potentially favorable delivery method to bypass the BBB and has been shown successful for transporting therapeutic agents directly from nose to brain along the olfactory and trigeminal nerves of the olfactory epithelium (Gupta et al., 2017; Kakadu et al., 2021; Mehrotra et al., 2023; Sarma et al., 2020). However, enzymatic degradation, entrapment by the nasal mucus followed by rapid mucociliary clearance, and possible efflux of such drugs by p-glycoprotein pumps are challenges that must be further addressed to make this route more feasible (Bourganis et al., 2018; Costa et al., 2021). Solid lipid nanoparticles (SLN) are biocompatible, biodegradable, and scalable nanocarriers that can alleviate many of these drawbacks of the N-t-B delivery route (Costa et al., 2021; Ghasemiyeh et al., 2018; Yasir et al., 2014). SLNs, when properly formulated, can load lipophilic drugs, protect them from enzymatic degradation and p-glycoprotein-induced efflux, and ensure high permeability of the drug across the nasal epithelium (Bourganis et al., 2018; Costa et al., 2021). However, their ability to permeate across the nasal mucosa can be obstructed due to their intrinsic lipophilicity that increases their interactions with the hydrophobic domains of the mucus layer, which thus traps and eventually clears such particles via mucociliary clearance (Alp et al., 2020; Gänger et al., 2018; Hua et al., 2024; Wang et al., 2008).

One approach that has been shown to overcome the mucus layer is the surface coating of nanoparticles with polyethylene glycol (PEG). PEG is a repeating unit of ethylene oxide that has been shown to have muco-penetrating abilities due to its hydrophilicity and neutral charge, which renders it muco-inert (Huckaby et al., 2018; Tafech et al., 2024). While PEGylation with high molecular weight PEG is known to induce muco-adhesion, studies demonstrate that both polymeric and lipid-based nanoparticles with densely coated, low molecular weight PEG exhibit favorable properties for muco-penetration and potentially N-t-B delivery (Correia et al., 2024; de Oliveira Junior et al., 2020; Kurano et al., 2022; Yuan et al., 2013). Additionally, polymeric nanoparticles with up to 10% (w/w) PEG surface coatings have been shown to enhance mucus penetration while keeping the nanoparticles hydrophobic enough to not hinder uptake by nasal epithelial cells. Thus, high-density, low-molecular-weight surface PEGylation may be a favorable modification to enhance the mucus penetration of nanoparticles, thereby improving the efficiency of intranasal drug delivery to the brain. However, there is currently no product on the market that exploits the favorable properties of PEGylated nanoparticles to improve the N-t-B delivery of therapeutic drugs or other agents (de Oliveira Junior et al., 2020; Food and Drug Administration (FDA), 2012).

The human immunodeficiency virus (HIV) is one of the deadliest and most prevalent infectious diseases in the world that primarily attacks the CD4+ T-cells of the immune system (Vidya Vijayan et al., 2017). If left untreated, a patient's T-cell count eventually drops below 200 cells/mm³, leading to the development of acquired immunodeficiency syndrome (AIDS). This severely weakened immune state makes patients highly susceptible to secondary infections, which can ultimately be fatal. There are currently 37.9 million people infected with HIV worldwide (The Global HIV/AIDS Epidemic, 2021), 1.2 million of which are in the United States (U.S. Statistics of HIV, 2021), with the mortality rate being over 90% if left untreated (Shelley A Gilroy, 2023).

In recent years, leaps and advancements have been made toward understanding and treating HIV and other viruses that infect humans and animals. To date, there are over 30 FDA-Approved antiretroviral therapies (ART) that can effectively suppress the viral load of the infected patient to an undetectable level when administered as a combined ART (cART) (Kaplan, 2020). However, ART must be taken every day for the rest of a person's life and when the risk of contracting is perceived to be high, ART can prevent infection in people without HIV either pre-exposure (PrEP) or post-exposure prophylaxis (PEP).

Although cART can successfully suppress the virus in the peripheral regions of the body, they are unable to sufficiently accumulate in the central nervous system (CNS) due to their low degree of permeability across the blood-brain barrier (BBB)(Osborne et al., 2020). This creates a haven for the virus, which in contrast, can cross the BBB and enter the CNS through several mechanisms, consequently infecting neuronal astrocytes, perivascular macrophages, and microglial cells (Killingsworth et al., 2022). The virus's ability to accumulate in the CNS can lead to various complications for infected patients, including rebound viremia if treatment ceases, thereby resuming the cycle of infection (Osborne et al., 2020). Moreover, HIV reservoirs in the brain induce a range of neurological dysfunctions, such as mania, depression, and HIV-associated dementia (HAD), collectively known as neuroAIDS (Saxena et al., 2013; Lindl et al., 2010; Wang et al., 2020). Thus, there remains a major gap in treatment that is an unmet need for treatment of this and other viral diseases and conditions.

### SUMMARY OF THE INVENTION

The invention comprises compositions and methods for preparing PEGylated solid lipid core nanocarriers (SLN) suitable for drug delivery to a subject in need thereof and methods for treatment with SLNs. The SLNs have a small size, neutral charge, and high drug encapsulation efficiency and are loaded with at least one therapeutic drug. PEGylation improves SLN nasal mucus permeability without hindering cellular uptake. Administration of the SLNs may be intranasal, inhalation, or injection. In particular, the PEGylated SLNs are suitable for intranasal drug delivery of a combined therapy, such as (cART) delivery to treat neuroAIDS.

In one embodiment, the invention is a nanocarrier enabled for delivery of at least one drug or therapeutic agent, comprising a solid lipid core and polyethylene glycol (PEG) chains attached to the surface of the core, wherein the at least one drug or therapeutic agent is encapsulated within the solid lipid core. In another embodiment, at least two drugs or therapeutic agents are encapsulated with the solid lipid core. In another embodiment, the at least two drugs or therapeutic agents are antiviral or antiretroviral drugs. In yet another embodiment, the antiretroviral drugs are atazanavir and elvitegravir.

In another embodiment, the invention is a method of preparing surface PEGylated solid lipid core nanocarriers comprising at least one drug or therapeutic agent, comprising the steps of: heating an oil phase solution comprising the at least one drug, polyethylene glycol and DMSO to 85° C. and concurrently heating an aqueous solution comprising an emulsifier to 85° C., placing the oil phase solution under continuous homogenization,

pouring the aqueous solution into the oil phase solution while continuing the homogenization for a suitable time period to obtain a hot oil in water (O/W) emulsion. This is followed by sonicating the O/W emulsion for a suitable time period to obtain an O/W nanoemulsion and then cooling the O/W nanoemulsion in an ice bath. In one embodiment, the at least one drug or therapeutic agent is an antiviral or antiretroviral drug.

In yet another embodiment, the invention is a method for treating AIDS in a subject in need thereof, comprising the steps of preparing surface PEGylated solid core nanocarriers (SCNs) comprising at least two antiretroviral drugs, and administering a therapeutically effective amount of the SCNs to the subject. In another embodiment, the AIDS is neuroAIDS. In yet another embodiment, the preparation of SCNs is administered intranasally.

Other features and advantages of the present invention will be set forth in the description of invention that follows, and in part will be apparent from the description or may be learned by practice of the invention. The invention will be realized and attained by the compositions and methods particularly pointed out in the written description and claims hereof.

### BRIEF DESCRIPTION OF THE DRAWINGS

The patent or application file contains at least one drawing executed in color. Copies of this patent or patent application publication with color drawings will be provided by the Office upon request and payment of the necessary fee.

The accompanying drawings, which are incorporated in and constitute a part of this specification, illustrate embodiments of the invention and, together with a general description of the invention given above, and the detailed description given below, serve to explain the invention.

FIG. 1 shows IR spectra of Pluronic F-127, PEG₁₀₀-LN, Compritol 888 ATO, ATZ, EVG, and their physical mixture.

FIG. 2 illustrates (Panel 2A) the relationship between cART-SLN PEGylation and nanoparticle size, and (Panel 2B) the relationship between cART-SLN PEGylation and PDI.

FIG. 3 shows scanning electron micrographs, wherein Panel 3A denotes the SEM image of CART-SLN_{PEG=0%}, while Panel 3B denotes the SEM image of cART-SLN_{PEG=15%}. The SLNs are depicted by the black particles present in the images.

FIG. 4 shows XRD Analysis of EVG, ATZ, CART-SLN_{PEG=0%}, and cART-SLN_{PEG=15%}.

FIG. 5 illustrates the stability of the C6-SLN formulations following dispersion in artificial nasal mucous (ANM) for 20 minutes at 37° C. The mean aggregate count per frame (n=5 frames) was analyzed via ImageJ software.

FIG. 6 shows representative confocal microscopy images of C6-SLN nanoparticles following a 20-minute incubation in ANM at 37° C. Image A shows nanoparticles with a PEGylation level of 0%; Image B shows nanoparticles with a PEGylation level of 15%.

FIG. 7 shows the mean aggregate size of C6-SLN formulations per frame (n=5 frames) following dispersion in ANM for 20 minutes at 37° C., analyzed by the ImageJ software. The total number of aggregates measured for each degree of PEGylation is the following: 0%=527, 5%=364, 10%=224, 15%=150.

FIG. 8 illustrates an assessment of the mass permeability of C6 across ANM over time for all four C6-SLN formulations with varying degrees of PEGylation, measured via a Transwell® system (n=3 Transwell® per formulation at each time point). A Transwell® system is an in vitro model used to measure nanoparticle permeability through the mucus. It consists of a mucus-coated porous membrane that separates the donor and acceptor compartments, allowing controlled evaluation of nanoparticle transport.

FIG. 9 shows the Pₐₚₚ of C6 across ANM for all four C6-SLN formulations with varying degrees of PEGylation, measured via a Transwell® system.

FIG. 10 shows fluorescent microscopy images of RPMI 2650 cells following 8 h incubation with C6-loaded SLNs at 37° C. Cell nuclei were labeled with DAPI (false-colored red). The presence of C6-SLNs in the cell cytoplasm was based on C6 fluorescence (colored green). Images A1-A3 display cells treated with C6-SLN_{PEG=0%}, while B1-B3 displays cells treated with C6-SLN_{PEG=15%}.

FIG. 11 shows a conceptual drawing of a surface PEGylated solid lipid nanocarrier dually loaded with molecules of two drugs. The nanocarrier comprises a solid lipid core with polyethylene glycol chains attached to the surface of the solid lipid core. In this drawing, the two drugs are identified as ATZ and EVG.

### DETAILED DESCRIPTION

The invention is a method for making and using PEGylated SLNs as a drug delivery vehicle. The SLNs enhance delivery of the drugs in a variety of ways. For example, the in vitro nasal mucus permeability of a therapeutic agent, such as a combination antiretroviral therapy (CART), is improved without hindering its cellular uptake as a vehicle for N-t-B delivery. In one embodiment of the invention, two HIV medications, elvitegravir (EVG), an integrase inhibitor, and atazanavir (ATZ), a protease inhibitor, are loaded in the SLNs as exemplary cART drugs due to their high potency and alignment with Food and Drug Administration (FDA) guidelines recommending their co-administration (FDA, 2012). Prior to this disclosure, there has been no formulation or method with the benefits of PEGylated SLNs for intranasal delivery of a cART to treat neuroAIDS.

In one embodiment, the invention is a nanocarrier enabled for improved drug delivery. The nanocarriers of the invention offer comprehensive benefits in pharmaceutical applications, including protection of drugs from enzymatic degradation and p-glycoprotein induced efflux, improvement of oral absorption, and facilitation of penetration of biological membranes, thus improving delivery of agents having maximum efficacy preserved. In particular, the nanocarriers are an improved pharmaceutical formulation comprising a PEGylated surface and a solid lipid core. A key benefit of the invention is the ability to combine administration and delivery of two or more drugs in a single nanocarrier. Thus, in one embodiment the invention is a nanocarrier dually loaded with two drugs that may be administered together as a single dose. The formulation combines and loads the drugs within the solid lipid core of the nanocarrier, which obviates the need to administer the drugs separately.

In one embodiment, the nanocarrier of the invention is a nanoparticle comprising a solid lipid core and polyethylene glycol (PEG) chains attached to the surface of the core. Nanoparticle surface PEGylation has multiple advantages. It has been shown to increase circulation time in the blood stream by minimizing recognition and clearance by the immune system. It also prevents aggregation of the solid lipid nanoparticle, increases mucus penetration in the stomach, lungs and nasal cavity, and can enhance the controlled release qualities of the drug-loaded solid lipid nanoparticle.

In another embodiment, the invention is a method of forming or preparing the nanocarriers, which are loaded with one or more drugs or active agents during the formation or preparation of the nanocarriers. In one embodiment, the method comprises formation of nanocarriers from a preparation of molten lipid phase of a polyethylene glycol mixed with a surfactant and the drug(s), wherein the mixture is homogenized and sonicated to achieve the desired particle size. The drug concentration within a nanocarrier is controlled by the amount of drug included in the mixture and by the final particle size.

In one embodiment the invention is a method of preparing and loading the nanocarriers with one or more drugs or active agents. In one embodiment, the nanocarriers may be loaded with two drugs or active agents. In another embodiment, the nanocarrier may be loaded with a plurality of drugs or active agents. In another embodiment, at least one of the drugs is an antiviral and/or antiretroviral drug or agent. In yet another embodiment, at least two of the drugs are antiviral and/or antiretroviral drugs.

In another embodiment, the invention is a method of treating a subject in need thereof by administering a plurality of the nanocarriers loaded with one or more drugs or active agents. The route of administration may include but is not limited to oral, intranasal, inhalation, or injection, including intraperitoneal, subcutaneous or intramuscular injection. The structure of the nanocarrier enhances cellular uptake and thereby improves targeting of the drugs. For oral administration, the nanocarriers may be contained within a capsule, such as a gelatin capsule, liquid dispersion, or dried or desiccated as a solid dosage form. Pharmaceutically acceptable excipients may also be added. For intranasal administration, the nanocarriers may be formulated as a nasal spray or applied directly to the nasal mucous membranes as a liquid suspension or dry powder of the nanocarriers. For inhalation, the nanocarriers may be formulated for administration via a nebulizer, or other liquid suspension atomizer, or dry powder device. For injection, the nanocarriers may be formulated in any pharmaceutically acceptable carrier.

As used herein, the terms “nanocarrier” and “solid core nanocarrier” or “SLN” are used interchangeably to refer to the surface PEGylated solid core nanocarriers of the invention. The SLNs are also referred to as “nanoparticles” in some instances.

As used herein, the term “polyethylene glycol” or “PEG” refers to a polyether compound also known as polyethylene oxide (PEO) or polyoxyethylene (POE), depending on its molecular weight. Pharmaceutical-grade PEG is used in many pharmaceutical products, including laxatives, hydrogels, excipients, and to PEGylate adenoviruses for gene therapy. The chemical structure of PEG is commonly expressed as H—(O—CH₂—CH₂)ₙ—OH, with a variable molecular weight depending on the number of ether (O—CH₂—CH₂) repetitions. For example, PEG 400 is a low-molecular weight PEG (having 9 ether repetitions and an average weight of 400 Daltons) frequently used to increase the solubility of a hydrophobic drug, while PEG 1500 is a high-molecular weight PEG that may be used as a water-soluble base. Thus, the molecular weight of the PEG may be chosen based upon the route of administration and barriers to uptake, such as mucus, tissues, cell membrane and other structures related to a biological target of the therapy. The molecular weight of PEG can vary widely depending on the specific type of PEG. Typically, PEG has a molecular weight ranging from 200 to 30,000 Dalton. Suitable molecular weights for forming the SLNs of the invention range from 400 Daltons to 10,000 Daltons. In one embodiment, the molecular weight is 600 Daltons to 9,000 Daltons. In another embodiment, the molecular weight is1,000 Daltons to 8,000 Daltons, 1,500 Daltons to 7,000 Daltons or 3,000 Daltons to 6,000 Daltons.

As used herein, the term “PEGylation” refers to the process of both covalent and non-covalent attachment of PEG polymer chains to a molecule or macrostructure, such as a drug, a therapeutic protein or a vesicle. PEGylation is known to improve the safety and efficacy of many therapeutics by producing desired alterations in conformation, electrostatic binding, hydrophobicity and other physiochemical properties. PEGylation can also alter pharmacological properties of drug solubility and stability, thus changing the dosage and safety profile of a drug. In practicing the method of the invention, PEGylation refers to the attachment of PEG polymer chains to the solid lipid core of a nanocarrier.

While the SLNs of the invention may be used to deliver a single drug or agent, they are particularly well-suited to deliver two or more drugs or agents that are intended for co-administration. The nanocarriers are able to deliver the drug combinations to the same target more effectively than when co-administered conventionally as a standard oral dosage form in two or more separate capsules. Because the SLNs may contain multiple drugs required by a dosing regimen, all of the drugs in an individual SLN are taken up by a target cell in a ratio that is controlled. This is important for drug regimens that rely on the actions of two or more drugs working in concert, particularly if the ratio of the two or more drugs is a critical factor. As an example, FDA guidelines stipulate that EVG and ATZ be administered together and are conventionally administered as two separate capsules in a standard oral dosage form. Loading molecules of ATZ and EVG into the nanocarriers effectively harnesses these combined dosing advantages.

Administration of the SLNs include but are not limited to the routes of intranasal, oral and intramuscular delivery. Using the example of ATZ and EVG, the loaded SLNs are suitable for an improved regimen for the treatment of HIV and neuroAIDS using any of these routes, since the current standard of care is oral administration of capsules. The SLNs improve targeting of the therapy via the ability to cross the blood-brain barrier and improved cellular uptake regardless of the route of administration. Thus, the SLNs are able to access viral reservoirs and inhibit reinfectivity of HIV.

Loading the nanocarriers with other antiviral drugs to target specific viruses is also contemplated, including acyclovir and/or valacyclovir for treatment of herpes simplex virus and varicella-zoster virus, oseltamivir for treatment of influenza, ganciclovir for treatment of cytomegalovirus, remdesivir for treatment of COVID-19 and efavirenz for treatment of HIV. Targeting of these antiviral drugs can also reduce their side effects, which may include nausea, vomiting, diarrhea, headache, dizziness and allergic reactions to the drugs. Additional benefits of treatments with the nanocarriers include but are not limited to reducing viral load, casing symptoms of a viral infection, reducing contagion to other subjects, reducing the length of a viral infection, and ameliorating neurological dysfunctions, such as mania, depression, and HIV-associated dementia (HAD). Thus, one embodiment of the invention is a method of relieving or reducing these aspects of viral disease or a condition associated with viral disease.

In addition to antiviral and antiretroviral drugs, main classes of HIV drugs may also be used for inclusion in the SLNs, including but not limited to protease inhibitors (PI), Integrase strand transfer inhibitors (INSTI), and non-nucleoside reverse transcriptase inhibitors (NNRTI). Additionally, some nucleoside reverse transcriptase inhibitors (NRTI), CCR5 antagonists, fusion inhibitors, and post-attachment inhibitors may be used as well, depending on their lipophilicity. Furthermore, combinations of antibiotics, peptide based antimicrobials, and/or metal nanoparticles may be delivered by the SLNs and these may be used in combination. All of these drugs can be used alone or in combination, pursuant to FDA guidelines and recommendations.

Other neurological conditions that may be treated using the SLNs include but are not limited to traumatic brain injury, epilepsy and depression. Thus, the drug(s) include but are not limited to a selective serotonin reuptake inhibitor (SSRI), a serotonin-norepinephrine reuptake inhibitor (SNRI), a monoamine oxidase inhibitor (MAOI), citalopram, escitalopram, fluoxetine, paroxetine, sertraline, desvenlafaxine, duloxetine, venlafaxine, bupropion, mirtazapine, methylphedinate, modafinil, amitriptyline, clomipramine, desipramine, doxepin, imipramine, nortriptyline, nefazodone, trazodone, vilazodone, isocarboxazid, phenelzine, tranylcypromine, agomelatine, escitalopram oxalate, fluvoxamine, protriptyline, citicoline, a statin, progesterone, erythropoietin, cyclosporine A, acetazolamide, brivaracetam, cannabidiol, carbamazepine, cenobamate, clobazam, clonazepam, eslicarbazepine acetate, ethosuximide, everolimus, fenfluramine, gabapentin, lacosamide, lamotrigine, levetiracetam, oxcarbazepine, perampanel, phenobarbital, phenytoin, piracetam, pregabalin, primidone, rufinamide, sodium valproate, stiripentol, tiagabine, topiramate, valproic acid, vigabatrin, zonisamide and combinations, thereof.

The SLNs are prepared by mixing a heated solution of a surfactant or emulsifier and a heated solution of PEG, DMSO and the drug(s) of interest. For the drug to successfully be encapsulated, the LogP must be positive, preferably at least 2.0; i.e., the drug is preferred to be lipophilic. The temperature is one sufficient to melt the solid lipid and PEG, which is typically about 85° C., but this temperature can vary depending on the structure of the lipid and molecular weight of the PEG and thus can be ±5° C. or more. The combining of the two solutions for a small-batch benchtop preparation is performed relatively slowly, for about 45 seconds, while under continuous homogenization. The combination of the two solutions is homogenized for an additional period of approximately 5 minutes. The homogenized solution is then sonicated for approximately 20 minutes at 65% or until the desired nanoparticle size is achieved. Depending on the volume, the homogenized solution may be subdivided into aliquots for sonication. Following sonication, the solution is rapidly cooled in an ice bath for approximately 30 minutes. One of skill in the art will recognize that these times will differ with larger batches, particularly when prepared at an industrial scale. In another embodiment, the nanoparticles are in the range of 10 to 350 nm. In one embodiment, the nanoparticles are equal to or smaller than 200 nm. In another embodiment, the nanoparticles are in the range of 10-200 nm, 20-180 nm, 30-160 nm or 50-150 nm.

The following descriptions and examples illustrate some exemplary embodiments of the disclosed invention in detail. Those of the skill in the art will recognize that there are numerous variations and modifications of this invention that are encompassed by its scope. Accordingly, the description of a certain exemplary embodiment should not be deemed to limit the scope of the present invention.

Before exemplary embodiments of the present invention are described in greater detail, it is to be understood that this invention is not limited to any particular embodiments described herein and may vary. It is also to be understood that the terminology used herein is for the purpose of describing particular embodiments only, and is not intended to be limiting, since the scope of the present invention will be limited only by the appended claims.

Where a range of values is provided, it is understood that each intervening value between the upper and lower limit of that range (to a tenth of the unit of the lower limit) is included in the range and encompassed within the invention, unless the context or description clearly dictates otherwise. In addition, smaller ranges between any two values in the range are encompassed, unless the context or description clearly indicates otherwise.

Unless defined otherwise, all technical and scientific terms used herein have the same meaning as commonly understood by one of ordinary skill in the art to which this invention belongs. Representative illustrative methods and materials are herein described; methods and materials similar or equivalent to those described herein can also be used in the practice or testing of the present invention.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual dates of public availability and may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as support for the recitation in the claims of such exclusive terminology as “solely,” “only” and the like in connection with the recitation of claim elements, or use of a “negative” limitations, such as “wherein [a particular feature or element] is absent”, or “except for [a particular feature or element]”, or “wherein [a particular feature or element] is not present (included, etc.) . . . “.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### Examples

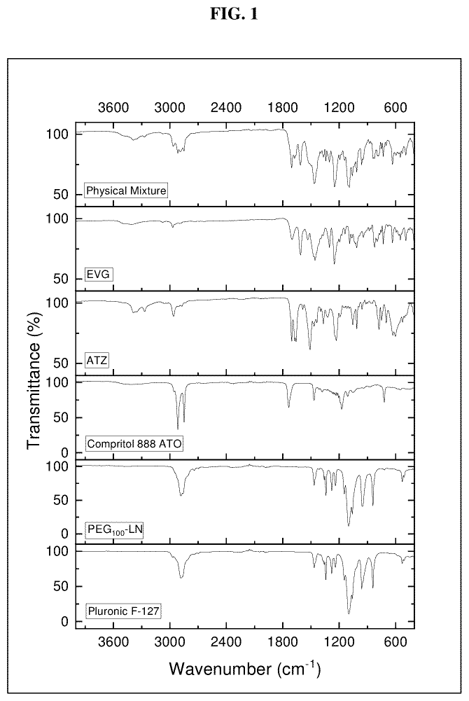

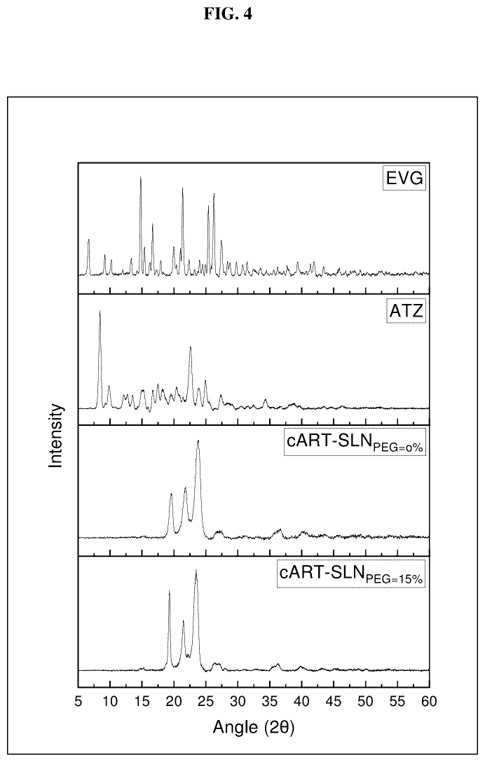

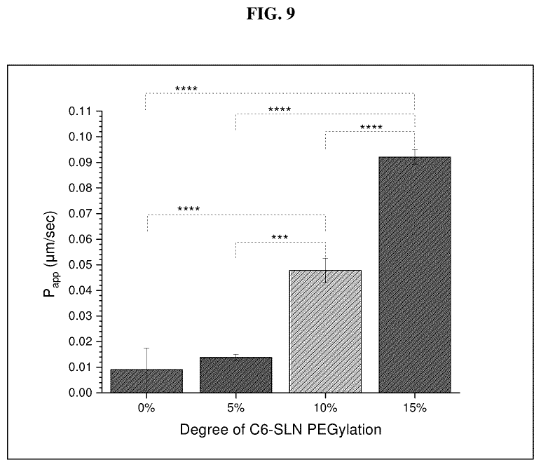

The following Examples provide exemplary compositions and methods for making and using the nanocarriers of the invention. These Examples describe materials and methods for using embodiments illustrated in FIGS. 1-11. Additional details can be found in the section entitled “Brief Description of the Drawings”.

Abbreviations cART, combination antiretroviral therapy; SLN, solid lipid nanoparticles; EVG, elvitegravir; ATZ, atazanavir; HIV, human immunodeficiency virus; AIDS, acquired immunodeficiency syndrome; FDA, Food and Drug Administration; ART, antiretroviral therapy; CNS, central nervous system; BBB, blood-brain barrier; HAD, HIV-associated dementia; N-t-B, nose-to-brain; PEG, polyethylene glycol; PEG₁₀₀-LN, PEG-100 stearyl ether; EC50, half maximal effective concentration; M.W., molecular weight; C6, coumarin-6; DMSO, dimethyl sulfoxide; ANM, artificial nasal mucus; FTIR, Fourier transform infrared spectroscopy; cART-SLN, CART-loaded SLN; cART-SLN_{PEG=0%}, CART-SLN containing 0% (w/w) PEG₁₀₀-LN; cART-SLN_{PEG=5%}, CART-SLN containing 5% (w/w) PEG₁₀₀-LN; CART-SLN_{PEG=10%}, CART-SLN containing 10% (w/w) PEG₁₀₀-LN; CART-SLN_{PEG=15%}, CART-SLN containing 15% (w/w) PEG₁₀₀-LN; PDI, polydispersity index; HPLC, high-performance liquid chromatography; R², correlation coefficient; DL, drug loading capacity; EE, encapsulation efficiency; M.W.C.O, molecular weight cutoff; SEM, scanning electron microscopy; XRD, X-ray diffraction; C6-SLN, SLN loaded with coumarin-6; C6-SLN_{PEG=0%}, C6-SLN containing 0% (w/w) PEG₁₀₀-LN; C6-SLN_{PEG=5%}, C6-SLN containing 5% (w/w) PEG₁₀₀-LN; C6-SLN_{PEG=10%}, C6-SLN containing 10% (w/w) PEG₁₀₀-LN; C6-SLN_{PEG=15%}, C6-SLN containing 15% (w/w) PEG₁₀₀-LN; PBS, phosphate-buffered saline; Pₐₚₚ, apparent permeability coefficient; EDTA, ethylenediaminetetraacetic acid; DAPI, 4′,6-diamidino-2-phenylindole

### Materials

The materials used in the experiments and their respective manufacturers are as follows: Compritol® 888 ATO was gifted by Gatefosse (Saint-Priest, France). PEG-100 stearyl ether (PEG₁₀₀-LN, PEG M.W.=4,405 Da) was purchased from Sigma Aldrich (St. Louis, Missouri, USA). Pluronic F-127 was gifted by BASF (Mississauga, Ontario, Canada). ATZ and Coumarin-6 were purchased from TCI Chemicals (Tokyo, Japan). EVG was purchased from Astatech (Bristol, Pennsylvania, USA). Dimethyl Sulfoxide (DMSO), acetonitrile, and fetal bovine serum were purchased from VWR (Radnor, Pennsylvania, USA). Phosphate-buffered saline was purchased from Corning (Corning, New York, USA). Artificial nasal mucus was purchased from Biochemazone (Leduc, Alberta, Canada). Lastly, the 0.25% (w/v) Trypsin-0.53 mM EDTA solution was purchased from Quality Biological (Gaithersburg, Maryland, USA).

### Fourier Transform Infrared Spectroscopy (FTIR)

Drug-excipient compatibility was determined using FTIR. Individual drugs, excipients, and their physical mixture were scanned using a Nicolet™ iS50 FTIR Spectrometer (Thermo Fisher Scientific Inc. United States) within the range of 400-4000 cm⁻¹.

### Preparation of Solid Lipid Nanoparticles

Per FDA guidelines, the recorded half maximal effective concentrations (EC50) of EVG and ATZ for HIV type-1 are 1.7 nM and 5 nM, respectively. After adjusting for their molecular weight, the EVG: ATZ dosing ratio is 1:4.63 (w:w), and was used for the formulation studies. The cART-loaded SLN (CART-SLN) was prepared using a modified melt-emulsification and ultrasonication method (Correia et al., 2024). Briefly, an oil phase containing 8.24 mg of ATZ, 1.78 mg of EVG, 0.5 mL of DMSO, and 324 mg of Compritol® 888 ATO/PEG₁₀₀-LN mixture (0, 5, 10, 15% (w/w) PEG₁₀₀-LN) was heated to 85° C. A separate aqueous solution containing 50 mL of Pluronic F-127 (1.5% w/v) as an emulsifier was concurrently heated to 85° C. The probe of the high-speed homogenizer (T-25 easy clean digital Ultra-Turrax®, IKA, Germany) was then placed in the beaker (100 mL capacity) with its tip positioned 0.25 cm above the oil phase. The homogenizer was turned on at 8000 RPM, and the aqueous phase was slowly added for 45 seconds to the oil phase. Homogenization continued for 5 minutes. The resulting hot oil in water (O/W) emulsion was then distributed into three separate vials (˜15 mL/vial), with each vial being sonicated (VCX 750, Sonics, USA) for 20 minutes at 65% amplitude using a 0.5-inch horn, positioned 2/3 deep into the emulsion. The temperature was maintained at 85° C. (±5° C.) throughout the preparation process. Afterward, the O/W nanoemulsion was rapidly cooled in an ice bath at 4° C. for 30 minutes, and drug-loaded SLNs were formed. The cART-SLN containing 0%, 5%, 10%, and 15% (w/w) PEG₁₀₀-LN was termed as cART-SLN_{PEG=0%}, CART-SLN_{PEG=5%}, CART-SLN_{PEG=10%}, and cART-SLN_{PEG=15%}, respectively. Alternatively, they were also referred to as cART-SLN with a degree or level of 0%, 5%, 10%, and 15% PEGylation, respectively.

### Characterization of Size, PDI, and Zeta Potential

Zetasizer (Malvern Panalytical, United Kingdom) was used to characterize the size, polydispersity index (PDI), and zeta potential of all SLNs. For both size and zeta potential analysis, 100 μl of the dispersion was diluted in 1,000 μl of NaCl solution (10 mM), and experiments were performed at 25° C. Size analysis was performed at a 90° angle of detection.

### High-Performance Liquid Chromatography

High-performance liquid chromatography (HPLC) (Shimadzu, Japan) equipped with an XBridge column (C18, 4.6×150 mm, 4.5 μm) was employed for the simultaneous quantification of EVG and ATZ. The system operated at 40° C. with a mobile phase flow rate of 1 mL/min, an injection volume of 20 μl, and a detection wavelength of 210 nm. The mobile phase was comprised of acetonitrile:water (55% acetonitrile, 45% water, v/v). Calibration was performed using standard solutions of EVG, ATZ, and their combined mixture, all dissolved in acetonitrile: DMSO (33% acetonitrile, 67% DMSO, v/v).

The retention times and peak characteristics of ATZ and EVG were first determined individually. Separate standard solutions for each drug were generated across concentrations ranging from 0.122-125 μg/ml. ATZ and EVG had retention times of approximately 4.2 minutes and 6.2 minutes, respectively (data not shown). The individual standard curves of both drugs exhibited high linearity (R²>0.999).

To evaluate consistency in their combined mixture, ATZ and EVG were then mixed in equal ratio to create the standard solutions. HPLC analysis confirmed that the retention times of ATZ (4.2 minutes) and EVG (6.2 minutes) remained consistent (data not shown). Standard curves of the two drugs generated from the mixture maintained a high linearity (R²>0.999) over the same concentration range listed above, for ATZ and EVG, respectively (data not shown). These standard curves were subsequently used for simultaneous quantification of both drugs.

### Measuring Drug Loading Capacity (DL) and Encapsulation Efficiency (EE)

DL and EE of all formulations were measured via the ultrafiltration method. Briefly, 1 mL of the nanosuspension was transferred to a Vivaspin® (Sartorius, Germany) centrifugal filter (3 KDa M.W.C.O), and centrifugation was performed for 12 hours at 4,000 RPM. The filtrate was subsequently sampled and diluted 1:1 with a 50:50 mixture of DMSO and acetonitrile to dissolve the unloaded drugs present in the aqueous phase. This solution was then analyzed using HPLC to determine the concentration of unencapsulated drugs, providing an indirect measurement of the individual EE for both EVG and ATZ, and their combined DL. The following formulas below define EE and DL:

$$\% EE = \frac{( W eight of drug added ) - ( W eight of free drug not encapsulated )}{( W eight of drug added )} \times 100 \qquad [ 1 ]$$

$$\% DL = \frac{( C o m bined weight of drugs encapsulated )}{( Total weight of nanoparticles )} \times 100 \qquad [ 2 ]$$

### Scanning Electron Microscopy (SEM)

To visualize its morphological properties, the SLN dispersion was diluted 1000-fold. Then, 5 μl was loaded on a copper grid and subsequently desiccated overnight. The desiccated samples were imaged by Hitachi SU-70 FE-SEM (Hitachi, Japan) at an operating voltage of 15 KV. Images were processed, with mean particle size measured using the ImageJ software.

### X-Ray Diffraction (XRD)

To help determine the crystalline or amorphous state of the two ARTs in the formulation, as well as their encapsulation, XRD Patterns of ATZ, EVG, and desiccated cART-SLN_{PEG=0%} and CART-SLN_{PEG=15%} formulations were measured using the Empyrean Multipurpose X-Ray Diffractometer (Malvern Panalytical, United Kingdom) with a revolving Cu anode, radiation source (1.54° A), performed at voltage 45 KV and current 40 mA. The samples were scanned between 5 and 60° (2θ), with a scan step size of 0.013°.

Coumarin-6 as a Fluorescent Model Drug for cART

For the in vitro mucus and cell studies, 1.75 mg of coumarin-6 (C6) replaced both drugs to track the nanoparticles. The same preparation method was used to create the SLNs loaded with coumarin-6 (C6-SLN) as with the cART. The DL and EE were measured via the ultrafiltration method against a standard curve (R²>0.999) of C6 dissolved in DMSO with concentrations ranging from 0.06-60 μg/mL, and measured via fluorescent plate reader (Victor Nivo, Perkin Elmer, USA) at an excitation and emission wavelength of 457 nm and 501 nm, respectively. The C6-SLN formulations containing 0%, 5%, 10%, and 15% (w/w) PEG₁₀₀-LN was termed as C6-SLN_{PEG=0%}, C6-SLN_{PEG=5%}, C6-SLN_{PEG=10%}, and C6-SLN_{PEG=15%}, respectively. Alternatively, they were also referred to as C6-SLN with a degree or level of 0%, 5%, 10%, and 15% PEGylation, respectively.

### In Vitro Release Studies of C6

To assess the stability of C6 as a fluorescent probe for nanoparticle tracking, the in vitro drug release profile of C6-SLN_{PEG=0%} and C6-SLN_{PEG=15%} were determined by using the dialysis bag diffusion method (Yasir et al., 2021). The release medium was comprised of PBS (PH˜7.35) with Pluronic F-127 (1.5% w/v) to enhance the solubility of C6. First, Slide-A-Lyzer™ dialysis cassettes (Thermo Fisher Scientific, USA), made of regenerated cellulose with a 7 kDa molecular weight cut-off, were immersed in the release medium for 15 minutes. Subsequently, 1.3 mL of each formulation (equivalent to 45 μg of C6) was inserted into the cassettes, and the cassettes were then submerged into a beaker containing 1000 mL of release medium, with temperature maintained at 37° C. The beakers were then sealed and kept on a magnetic stirrer at 100 rpm. The medium was sampled at time intervals of 0.5, 1, 2, 4, 6 8, 12, and 24 hours, and replaced with fresh medium to maintain sink conditions. Samples were analyzed via fluorescent plate reader against a standard curve ranging from 0.0025-0.078 μg/mL, with an R²>0.999. Both formulations were tested in triplicates.

### Mucus Stability Studies

The stability of each C6-SLN formulation in artificial nasal mucus (ANM) was analyzed via confocal microscopy. Specifically, 100 μL of C6-SLN was dispersed in 1000 μL of ANM for 20 minutes at 37° C. Following incubation, five images of each formulation were promptly captured using a confocal microscope (Zeiss LSM 880, Zeiss, USA) at a magnification of 40-fold. The captured images of each formulation then underwent analysis to calculate its average aggregate count and size using the ImageJ software.

Mucus penetration test using a Transwell® system: The permeability of C6-SLNs through the artificial nasal mucus layer was assessed using a 24-well Transwell® insert (Corning®, USA). Initially, 50 μl of ANM was uniformly applied to the donor compartment of each well. The acceptor compartment was filled with 0.6 mL of phosphate-buffered saline (PBS). Subsequently, 200 μl of each formulation containing varying degrees of PEGylation was introduced to the donor compartment. Samples were collected from the acceptor compartment at intervals of 0.5, 1, 1.5, 2, 2.5, and 3 hours. These samples were analyzed for their fluorescence intensity using a plate reader set to an excitation and emission wavelength of 457 nm and 501 nm, respectively.

### Measuring the Apparent Permeability Coefficient

The apparent permeability coefficient, Pₐₚₚ, of all permeability studies listed above was calculated by the following formula:

$${P}_{a p p} = {K}_{d} \times \frac{1}{A \times {C}_{0}} \qquad [ 3 ]$$

Whereas K_{d} represents the slope of the linear portion in the mass of C6 permeated into the acceptor compartment, measured between 2.5 and 3 hours. Additionally, A is the area of the Transwell® (0.33 cm²), and C₀ is the initial concentration of C6 in the formulation (35 μg/mL).

### RPMI 2650 Nasal Epithelial Cell Culture

The RPMI 2650 nasal epithelial cells were obtained from the American Type Culture Collection (Catalog number CCL-30™, lot number 70045104) and used from passages 3-15. The cells were maintained in MEM (Eagle's Minimum Essential Medium, Corning®, USA) supplemented with sodium bicarbonate (1.5 g/L), non-essential amino acids, L-glutamine, sodium pyruvate, and 10% (v/v) fetal bovine serum. The cell cultures were maintained at 37° C. in a >95% humidified atmosphere of 5% CO₂ in the air with media changes every two days.

### Imaging the In Vitro Cellular Uptake of C6-SLNs

Once the cells reached 70-80% confluency, they were incubated for 15 minutes with 0.25% (w/v) Trypsin-0.53 mM EDTA solution, detached, and seeded on 24-well imaging plates at a density of 1.18×10⁶ cells per well. Following three days of seeding, each well was treated with 100 μg of C6-SLNs containing varying degrees of PEGylation (0% and 15%) for 8 hours at a temperature of 37° C. Subsequently, the cells were rinsed three times with 2 mL of PBS and then fixed with a solution of cold methanol (1 mL). The nuclei were then stained using DAPI (4′,6-diamidino-2-phenylindole, 2 μg/mL), and uptake of the nanoparticles was observed using an inverted fluorescence microscope (Zeiss Axio Observer Z1, Zeiss, USA) at a 20-fold magnification.

### Quantifying the In Vitro Cellular Uptake of C6-SLNs

RPMI 2650 cells were seeded in 24-well plates at a density of 1.18×10⁶ cells per well. Three days post-seeding, each well (n=4 wells per formulation) was treated with 100 μg of C6-SLN formulations containing varying degrees of PEG (0% & 15%) for 8 hours at a temperature of 37° C. Subsequently, the cells were rinsed three times with PBS (2 mL), trypsinized, and fluorescent uptake of C6-SLNs was quantified via Invitrogen Countess 3 FL Automated Fluorescent Cell Counter (Thermo Fisher, USA).

### Statistical Analysis

Statistical data analysis was performed using Tukey's honest significance test with p<0.05 as the minimal level of significance. The results were expressed as mean±standard deviation (SD) obtained from a minimum of three separate samples (n≥3). All error bars shown in the figures denote the standard deviation. A single asterisk (*) between two groups in a figure indicates a p-value less than 0.05, two asterisks (**) indicate a p-value less than 0.01, three asterisks (***) indicate a p-value less than 0.001, and four asterisks (****) indicate a p-value less than 0.0001. All statistical analysis and their corresponding figures were generated using the OriginPro software (OriginLab Corporation, USA).

### Results

This example demonstrates the design of SLNs as carriers for nose-to-brain delivery of CART and the benefits of surface PEGylation on nasal mucus penetration. The chemical interactions between drugs and excipients were characterized to ensure no reactions were occurring that might compromise the efficacy of the antiretroviral therapies. Once this was confirmed, formulation studies characterized cART-SLNs with varying degrees of PEGylation. We assessed (A) their DL and EE into the SLN matrix, which determines the required administration volume; (B) zeta potential, which affects mucus and epithelial interactions; (C) size, which influences mucus permeability and cellular transport; (D) morphology, which confirms the size and shape of the nanoparticles; and (E) crystallinity, which confirms cART encapsulation. After characterizing these attributes, we demonstrated the impact of PEGylation on the nasal mucus stability, permeability, and cellular uptake of SLNs by using C6, a fluorescent model drug, as a substitute for cART to track the SLNs within these environments. Increasing PEGylation levels enhance the SLN penetration across the nasal mucus layer and, in turn, increase the overall absorption by nasal epithelial cells.

### FTIR Analysis

Pre-formulation studies to detect chemical interactions between the drugs and excipients via FTIR are shown in FIG. 1. For ATZ, an NH stretch appears at 3389.56 cm⁻¹, 3268.64 cm⁻¹ due to OH stretching, 2960.76 cm⁻¹ due to a CH stretching, 1705.05 cm⁻¹ due to a C═O stretching, 1510.12 cm⁻¹ due to C═C stretching, and 1231.26 cm⁻¹ due to C—O—C stretching. For EVG, a peak appeared at 3405.47 cm⁻¹ due to an OH stretch, 2969.69 cm⁻¹ due to a CH stretch, 1701.55 cm⁻¹ and 1610.81 cm⁻¹ due to a C—O stretch, 1456 cm⁻¹ due to a C═C stretch, and 1251.40 due to a CO stretch. All of these characteristic peaks for both drugs are present in the physical mixture without any significant shift, and the additional peaks are attributed to the presence of excipients. This suggests that no chemical interaction occurs between the drugs and excipients that would alter the structure of EVG or ATZ, indicating their compatibility. Previous studies with various drugs and excipients have consistently reached similar conclusions, as reported in the literature (Akel et al., 2021; Bakshi et al., 2022; Hasan et al., 2021; Saini et al., 2021).

Size, Charge, PDI, pH, Drug Loading, and Encapsulation Efficiency of cART-SLN

A comprehensive overview of all four cART-SLN formulations is shown in Table 1, with levels of PEGylation ranging from 0% to 15%, sterically stabilized by Pluronic F-127. Since the nasal drug administration volume is typically limited to 150-200 μL per nostril in adults, it is critical to maximize the DL and EE of our cART-SLN formulation to ensure adequate dosing (Alabsi et al., 2022; Chung et al., 2023). Here, the EE for all formulations surpassed 99% for both drugs, and each maintained a consistent DL of 3%. Without being bound by theory, this high encapsulation is likely attributed to the significant lipophilicity of EVG and ATZ (LogP>4.0), which promotes effective incorporation into the lipid matrix of SLNs. Furthermore, the zeta potential of all formulations remains relatively neutral (>−5 mV). A neutral zeta potential is an important parameter, as the nasal mucus is negatively charged (Beule, 2010). Thus, keeping the charge of the nanoparticles neutral minimizes its interactions with the mucus. Additionally, the pH of CART-SLN_{PEG=0%} and cART-SLN_{PEG=15%} were 5.7±0.118 and 6.16±0.042, respectively, which indicates that all four formulations have a pH that ranges within the physiological conditions of the nasal mucosa (England et al., 1999).

TABLE 1
Comprehensive summary of size, PDI, zeta potential, EE, and combined
DL for all four cART-SLNs with degrees of PEGylation ranging from
0%-15%. SLN concentration for all formulations was 6.42 mg/ml.

| Degree of | | | Zeta | | | |
|---|---|---|---|---|---|---|
| cART-SLN | Size | | potential | EE of | EE of | Combined |
| PEGylation | (nm) | PDI | (mV) | EVG | ATZ | DL (w/w) |
| 0% | 59.24 ± 4.65 | 0.219 ± 0.029 | −3.28 ± 0.42 | >99% | >99% | 3% |
| 5% | 55.02 ± 1.98 | 0.232 ± 0.014 | −2.29 ± 0.45 | | | |
| 10% | 53.34 ± 2.12 | 0.233 ± 0.020 | −3.83 ± 0.97 | | | |
| 15% | 52.15 ± 3.57 | 0.268 ± 0.018 | −4.39 ± 0.97 | | | |

Particle size is also a critical factor for intranasal delivery, as nanoparticles smaller than 200 nm have demonstrated the most favorable permeability across the nasal mucosa (Samaridou et al., 2018). This is likely due to the average diameter of neuronal axons in the olfactory region being approximately 200 nm, which sets a morphological size constraint for the intraneuronal transport of nanoparticles to the brain (Mistry et al., 2009; Samaridou et al., 2018). Furthermore, the mesh space of mucins in the mucus layer also has an upper limit of 200 nm, which can hinder the diffusion of larger particles (Gänger et al., 2018; Samaridou et al., 2018). Therefore, having a nanoparticle smaller than 200 nm is preferred.

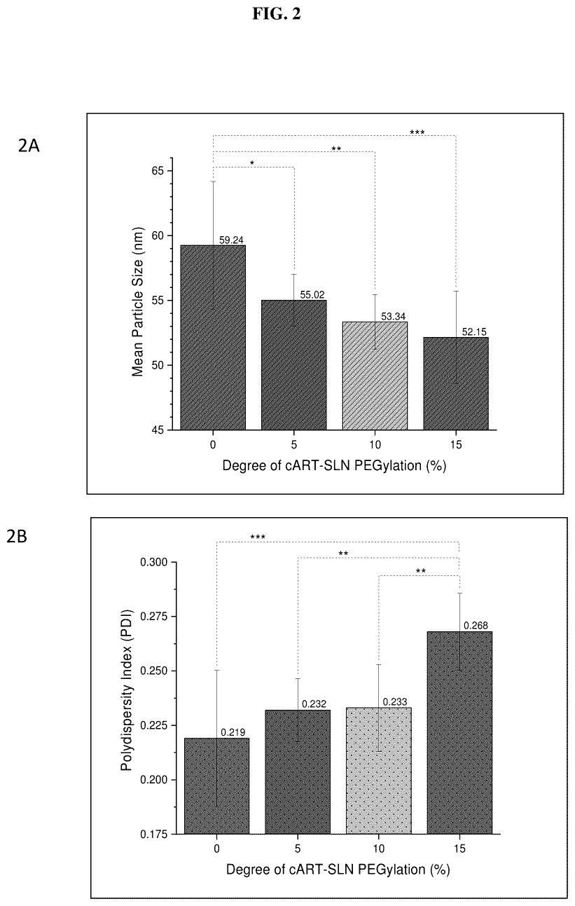

Panel 2A of FIG. 2 demonstrates that the mean nanoparticle size for all four formulations was less than 60 nm. Further comparison of non-PEGylated and 5, 10, and 15% PEGylated formulations shows a statistically significant linear decrease in mean particle size by increasing PEGylation levels, with the reduction becoming notable at 5% PEGylation (p-value <0.05) and more pronounced at 10% and 15% PEGylation levels (p-value <0.01). This size reduction is likely due to the steric repulsion from PEGylation, which prevents inter-particle interactions and further enhances nanoparticle stability by reducing aggregation during formation (Shi et al., 2021).

Panel 2B of FIG. 2 shows that increasing PEGylation to 5% and 10% did not significantly impact the PDI. However, a further increase to 15% PEGylation resulted in a statistically significant rise in PDI compared to the 0, 5, and 10% PEGylated formulations (p-value <0.01). The observed increase in PDI suggests that PEGylation introduces a degree of heterogeneity in particle size distribution. This heterogeneity may partly arise from variations in both the PEG chain lengths and their surface densities on nanoparticles, which can affect the overall distribution of particle morphology within the dispersion (Rabanel et al., 2019). While PEGylation appears to affect the size and PDI of cART-SLNs, all formulations produce relatively monodispersed nanoparticles (PDI<0.3) that are smaller than the pore sizes of nasal mucus (100-200 nm) and the neuronal axons of the olfactory epithelium. These characteristics make them well-suited for diffusion through the nasal mucus and intraneuronal transport.

### Scanning Electron Microscopy

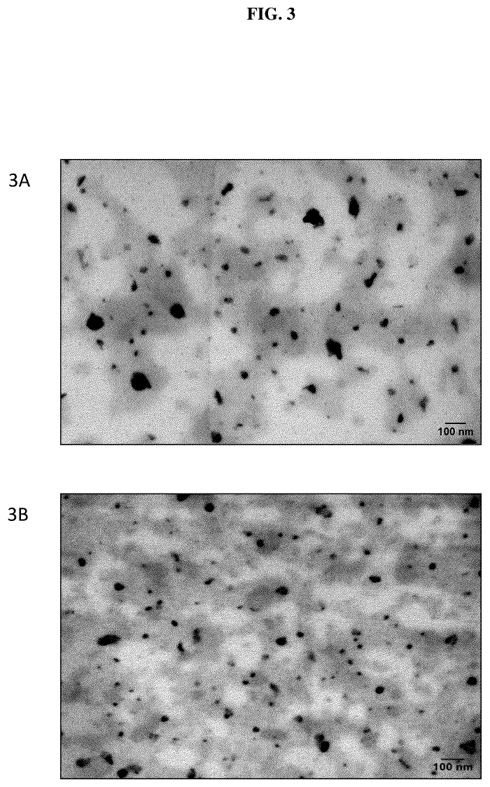

The SEM results display the cART-SLNs as the black particles shown in FIG. 3. Analysis revealed that both the non-PEGylated cART-SLNs (see Panel 3A) and those with a 15% PEGylation level (see panel 3B) maintained a primarily spherical morphology, but Panel 3B implies that PEGylation may enhance the consistency in spherical morphology. Size distribution analysis of the images revealed that non-PEGylated cART-SLNs (n=57) had particle sizes ranging from 16 to 78 nm, with a mean size of 31.5 nm and a standard deviation of 15.4 nm. In comparison, nanoparticles with 15% PEGylation (n=62) exhibited a narrower size range of 16 to 41 nm, with a mean size of 23.0 nm and a standard deviation of 6.4 nm.

The smaller mean particle sizes observed in SEM compared to those measured by the Zetasizer can be attributed to the differences in measurement techniques. The Zetasizer utilizes dynamic light scattering (DLS) to determine the hydrodynamic diameter of nanoparticles in a dispersion. This method accounts for the size of the core particle, as well as the hydration layer and any adsorbed molecules (such as surfactants), which can result in a larger recorded size (Fissan et al., 2014). In contrast, SEM directly visualizes the physical dimensions of dried nanoparticles, providing a more precise measurement of the core particle size.

As detailed above, SEM analysis also showed a more uniform particle size distribution for the cART-SLNs with a level of 15% PEGylation compared to the non-PEGylated ones. This again deviates from the DLS results, where PEGylation at this level increased the PDI. The greater size uniformity of the PEGylated SLNs, observed through SEM, may be attributed to the stabilizing effects of PEGylation throughout sample preparation. PEG coating induces steric repulsion between nanoparticles, which can minimize their interactions as water loss concentrates them closer together (Kakkar et al., 2015; Shi et al., 2021). SLN PEGylation may have thus prevented nanoparticle aggregation during the desiccation step before imaging, ensuring increased uniformity.

### XRD Analysis

As shown in 4, the X-ray diffractograms of ATZ and EVG exhibited sharp peaks at various angles, which designate the crystalline structure of both drugs. Notable peaks for EVG are shown at 6.71°, 14.82°, 21.36°, 25.38°, and 26.28°. For ATZ, the most prominent peak is at 8.41° followed by 22.55°. However, for the diffractograms of both cART-SLN_{PEG=0%} and cART-SLN_{PEG=15%} formulations, only three distinct peaks were shown at specific angles, and neither displayed the collection of characteristic peaks for the ATZ or EVG crystals mentioned above. These findings confirm that both drugs were encapsulated in their amorphous form within the lipid matrix of both cART-SLN_{PEG=0%} and cART-SLN_{PEG=15%} formulations. Previous studies using different drugs and lipid-based carriers have consistently drawn similar conclusions, as documented in the literature (Al-sayadi et al., 2024; Bunjes et al., 2007; Prajapati et al., 2021; Yasir et al., 2021).

### Characterization of Coumarin-6 (C6) as a Fluorescent Model Drug

C6 was selected as a fluorescent model drug in place of both EVG and ATZ to monitor the diffusivity and stability of nanoparticles within the artificial nasal mucus. This choice was determined by C6 having similar physicochemical characteristics to the ARTs, including a LogP greater than 4 and a molecular weight under 1000 Da. Furthermore, C6 exhibits rapid fluorescence loss in aqueous environments due to its propensity to form nanocrystals as a result of its poor water solubility (Banerjee et al., 2017). Its fluorescence is thus primarily observable when encapsulated within the SLN matrix, providing a reliable probe for nanoparticle tracking.

A detailed summary of the four C6-SLN formulations is shown in Table 2, which also vary in PEGylation levels ranging from 0% to 15%. Overall, the physicochemical properties of these C6-SLNs closely resemble those of their corresponding cART-SLN formulations. Key similarities include a mean nanoparticle size range of 45-60 nm, PDI values between 0.2-0.3, a relatively neutral nanoparticle charge (>−5 mV), and an entrapment efficiency of over 99%.

In vitro drug release studies revealed that for up to 24 hours, no quantifiable mass of C6 was released into the media for both C6-SLN_{PEG=0%} and C6-SLN_{PEG=15%} nanoparticles. This indicates a C6 release profile of less than 6% from the SLNs for both formulations by 24 hours, based on the lower limit of the standard curve. Although the full release profile could not be measured within this time frame, these results reinforce the notion that, for the subsequent studies involving C6, the lipophilic fluorescent model drug likely remained loaded within the SLNs, effectively probing the nanoparticles.

TABLE 2
A comprehensive summary of size, PDI, zeta potential, DL, and EE
for all four C6-SLNs with degrees of PEGylation ranging from 0%-
15%. SLN concentration for all formulations was 6.42 mg/ml.

| | | | Zeta | | |
|---|---|---|---|---|---|
| Degree of C6- | Size | | potential | DL | |
| SLN PEGylation | (nm) | PDI | (mV) | (w/w) | EE |
| 0% | 51.67 ± | 0.187 ± | −2.30 ± | 0.54% | >99% |
| | 0.20 | 0.011 | 0.26 | | |
| 5% | 50.63 ± | 0.201 ± | −4.11 ± | | |
| | 0.07 | 0.012 | 0.21 | | |
| 10% | 47.57 ± | 0.175 ± | −2.91 ± | | |
| | 0.11 | 0.004 | 0.45 | | |
| 15% | 50.55 ± | 0.232 ± | −4.69 ± | | |
| | 0.62 | 0.012 | 0.93 | | |

### Mucus Aggregation Studies

Following intranasal administration, the first barrier that SLNs interact with is the environment of the nasal mucus, which is predominantly composed of water, ions, and mucins (Lai et al., 2009; Williams et al., 2006). The interaction between nanoparticles and the mucosal components is critical, as mucin adsorption onto the nanoparticle surface can induce significant changes including colloidal destabilization, a process that is characterized by particle aggregation that leads to an increase in particle size (Xu et al., 2015). Such changes can significantly reduce the mucus permeability of these nanoparticles due to the small mesh space (<200 nm) of mucins in the nasal mucus (Ganger et al., 2018; Samaridou et al., 2018). Thus, to assess the stability of C6-SLNs, we incubated them in ANM for 20 minutes and monitored their aggregation behavior.

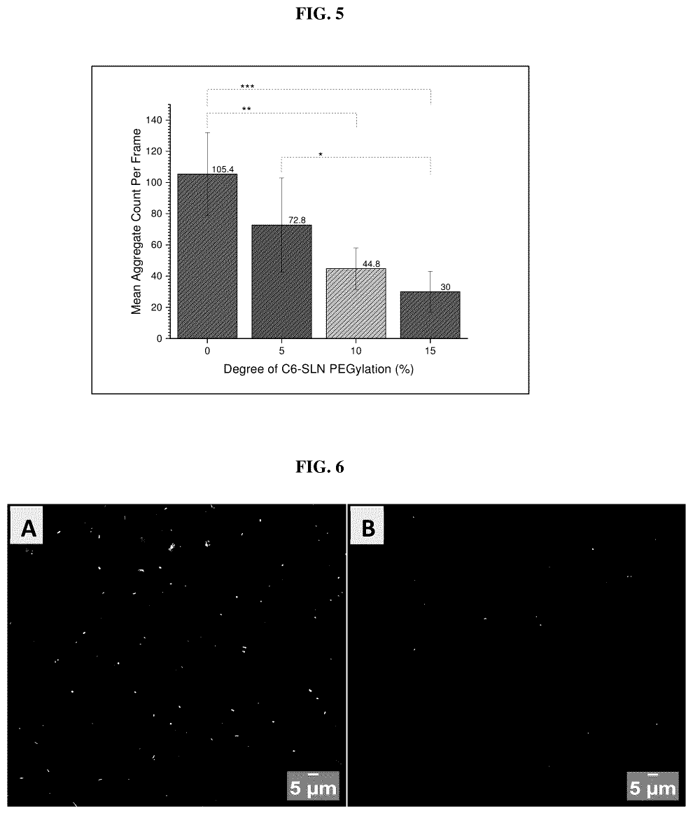

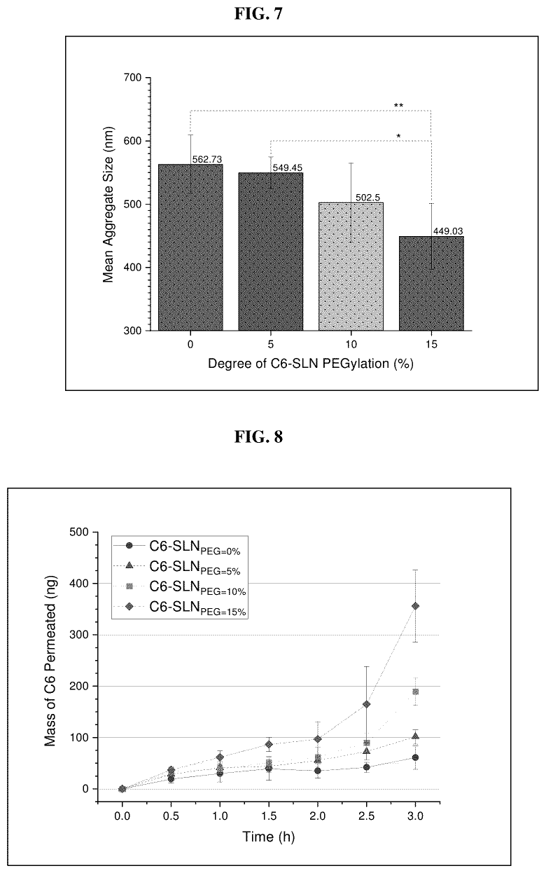

As illustrated in FIG. 5, we quantified the nanoparticles that aggregated into clusters larger than 200 nm across various samples. After incubation, the number of nanoparticle aggregates in C6-SLN_{PEG=5%}, C6-SLN_{PEG=10%}, and C6-SLN_{PEG=15%} decreased by 1.44-fold, 2.35-fold, and 3.51-fold, respectively, compared to the non-PEGylated C6-SLNs, which can be visualized in FIG. 6. Additionally, FIG. 7 demonstrates a consistent reduction in the average size of aggregates with progressive increases in PEGylation levels to 5%, 10%, and 15%. These trends underscore the stabilizing influence of PEGylation on nanoparticle aggregates in the nasal mucus, indicating enhanced stability with increasing PEGylation levels.

The instability observed in the non-PEGylated C6-SLN formulation can be attributed to excessive mucin deposition on the nanoparticle surfaces, which PEGylation can avoid depending on the surface density and the molecular weight of PEG used (Bertrand et al., 2017; Pelaz et al., 2015). Mucin, a key protein in the nasal mucus, binds to nanoparticles through electrostatic and hydrophobic attractions, as well as hydrogen bonding, leading to nanoparticle aggregation (Ensign et al., 2012; Ying-Ying Wang et al., 2008). Hydrophobic surfaces, which do not form hydrogen bonds with water, disorder the water's hydrogen-bonded network, leading to increased adhesion between each other's surfaces. This adhesion results in decreased mobility of water molecules, which is energetically unfavorable as it reduces the entropy of the system. However, PEGylation with at least 5% PEG-LN alters this by adding PEG groups that can bond with water via hydrogen bonding without reducing entropy, thus minimizing hydrophobic interactions and making them more energetically favorable (Ying-Ying Wang et al., 2008). These findings are corroborated by Xu et al. (Xu et al., 2015) who also demonstrated that low molecular weight PEG at a density of 5% was able to prevent the nanoparticle from interacting with the mucus, due to its improvement in the stability of the nanoparticle by increasing its colloidal stability, reducing its zeta potential, and overall, reducing the hydrophobic interactions with the mucus (Huckaby et al., 2018; Maisel et al., 2016).

### Mucus Permeability Studies

Mucociliary clearance is a process where the mucus layer traps and clears particles from the nasal cavity within a short time frame, which means that a drug must diffuse through it beforehand to reach the nasal epithelial cells for absorption. It is thus suggested that effective nanoparticle formulations for intranasal delivery should swiftly permeate the drug through the nasal mucus to minimize the effects of mucociliary clearance (Gänger et al., 2018). This is supported by recent studies, which report that nanoparticles enhancing a drug's diffusivity within the nasal mucus effectively improve its transport to the brain (de Oliveira Junior et al., 2020). We therefore measured the ability of the four C6-SLN formulations to enhance the permeability of C6 using a Transwell® system. 8 presents the findings, which indicate that PEGylation of nanoparticles led to a significant increase in the mass of C6 that permeated through the ANM layer after three hours. Specifically, compared to the non-PEGylated formulation, the mass of C6 that permeated increased by 1.6-fold, 3.15-fold, and 5.83-fold for C6-SLNs with a degree of 5%, 10%, and 15% PEGylation, respectively.

Another key method to assess how SLN PEGylation enhances the permeability of an encapsulated drug across the nasal mucus is by calculating its Pₐₚₚ, which measures the speed at which a drug travels through a biological barrier at steady state (Butnarasu et al., 2023). As shown in FIG. 9, the Pₐₚₚ of C6 across the ANM significantly increased when loaded in the PEGylated SLNs, indicating enhancements of 1.5-fold, 5.2-fold, and 10.1-fold with a degree of 5%, 10%, and 15% PEGylation, respectively, compared to the non-PEGylated C6-SLN formulation. At a degree of 0% PEGylation, C6 has a Pₐₚₚ of 0.00914 μm/sec, while at 15% PEGylation, the Pₐₚₚ is 0.09210 μm/sec. The nasal mucus in the human body is approximately 10-15 μm thick, with the time for nasal mucociliary clearance estimated to range from 4-9 minutes (X. Gao et al., 2023). Taking these constraints into account, the C6 loaded in the non-PEGylated SLN would only travel 2.16 μm by 4 minutes and 4.93 μm by 9 minutes, which indicates that it would not cross the thickness of the nasal mucus layer before mucociliary clearance is complete. However, the C6 loaded in the C6-SLN_{PEG=15%} formulation will theoretically travel through 22.11 μm of mucus in 4 minutes, which means that it can completely penetrate through the mucus layer before mucociliary clearance takes place and can hence reach the nasal epithelial cells for uptake and transport. These results demonstrate that PEGylation of SLN enables the permeability of encapsulated drugs, such as cART, across the nasal mucus due to its ability to increase the rate of permeability. These factors are once again attributed to the neutral charge and hydrophilicity of PEG that reduces their hydrophobic and ionic interactions with the mucus, their steric hindrance that prevents the binding of nanoparticles to the mucin fibers, and the reduction in nanoparticle aggregation that allows them to pass through the pores of the mucus.

### Cell Uptake Studies

Surface PEGylation is an increasingly popular modification to nanoparticles due to its well-documented ability to extend their systemic circulation half-life (Y. Gao et al., 2024; Suk et al., 2016). This is achieved by forming a protective hydrophilic barrier around the particle, which sterically hinders protein adsorption. The steric hindrance prevents the mononuclear phagocytic system from recognizing and binding to the nanoparticles, thereby reducing their clearance rate from the body (Verhoef et al., 2013). While this characteristic is beneficial for increasing circulation time, there is ongoing concern about whether PEGylation might negatively impact the uptake and internalization by target cells. This concern has become increasingly evident in the field of gene delivery, where studies demonstrate that PEGylation inhibits transfection rates due to reduced internalization (Knop et al., 2010). Therefore, we needed to determine the impact of PEGylation on the uptake of SLNs by the nasal epithelium. This is important because SLN internalization occurs through an active process called endocytosis, which is mediated by proteins such as clathrin and caveolin (Al Khafaji et al., 2021). Consequently, PEGylation's ability to decrease protein adsorption could play a critical role in hindering this process.

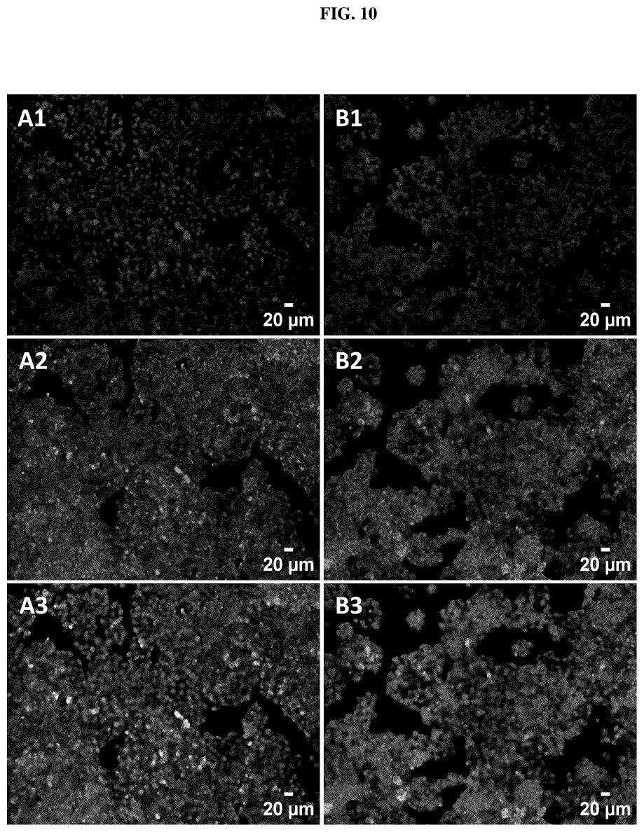

The effect of PEGylation on SLN uptake by nasal epithelial cells was determined using the immortalized RPMI 2650 cell line, which closely resembles normal human nasal epithelium and is thus commonly used in studies of nasal uptake and permeability (de Oliveira Junior et al., 2020; Schlachet et al., 2019; Sibinovska et al., 2019). Using visual analysis via fluorescent microscopy as shown in FIG. 10 the study demonstrated that after 8 hours of cellular incubation with the SLNs, there were no noticeable differences in the cellular uptake of both non-PEGylated and PEGylated C6-SLNs. Quantitative analysis via automated fluorescent cell counter demonstrated that non-PEGylated SLNs had an average uptake rate of 99.5%, and SLNs with 15% PEG had an average uptake rate of 100%, without any statistically significant difference (p-value >0.05).

Despite suggestions that a PEG corona might reduce nanoparticle uptake due to steric interactions with the cell membrane (Li et al., 2014), our findings demonstrate that coating our SLNs with up to 15% PEG did not deter their uptake by the RPMI 2650 cells following 8 hours of exposure. This could be due to the neutral zeta potential of the PEGylated SLNs, which has been shown to enhance nanoparticle internalization (Samaridou et al., 2020). Additional attributions to the unhindered uptake can be the high grafting densities of low molecular weight PEG on the surface of SLN, which facilitates cellular uptake through weak hydrophobic interactions or hydrogen bonding between the distal methoxy groups of the PEG chains and cell membranes (de Oliveira Junior et al., 2020; Li et al., 2014; Pelaz et al., 2015).

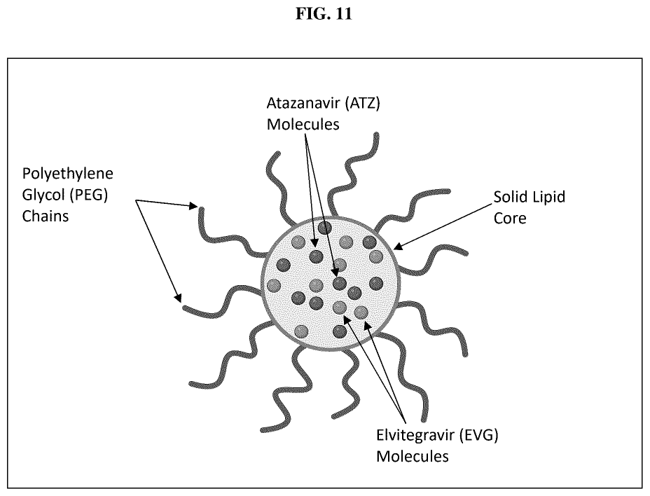

Thus, PEGylated SLN formulations can effectively penetrate the nasal mucus and be internalized by nasal epithelial cells after intranasal administration. In recent years, nose-to-brain delivery of therapeutics by the use of nanoparticles, specifically SLNs, has become an emerging and favorable delivery route for the treatment of various neurological diseases, including neuroAIDS. To harness the potential advantages of nose-to-brain delivery for this disease, we loaded cART into SLNs, characterized their physiochemical properties, and determined that their size, charge, PDI, loading, and encapsulation efficiency are very favorable for intranasal delivery applications. FIG. 11 shows a conceptual drawing to illustrate the encapsulated cART comprising ATZ and EVG withing the solid lipid core. We further demonstrated the advantages of PEGylated cART-SLN formulations for penetrating the nasal mucus, one of the major barriers for intranasal delivery of drugs. Comparing four different levels of PEGylation, we characterized that adding up to a level of 15% PEGylation (w/w of PEG₁₀₀-LN) to the SLN significantly increases the encapsulated model drug's rate of mucus permeability by increasing nanoparticle stability in the mucus, yet concurrently, without hindering uptake by nasal epithelial cells. These findings demonstrate that PEGylated SLNs as a platform can be used for the intranasal delivery of therapeutic drugs or other agents, including cART for nose-to-brain delivery to treat neuroAIDS.

While the invention has been described in terms of its several exemplary embodiments, those skilled in the art will recognize that the invention can be practiced with modification within the spirit and scope of the appended claims. Accordingly, the present invention should not be limited to the embodiments as described above but should further include all modifications and equivalents thereof within the spirit and scope of the description provided herein.

### REFERENCES

Akel, H et al. (2021). Int J Pharmaceu, 604, 120724.doi: 10.1016/j.ijpharm.2021.120724
Alabsi, W et al. (2022). Pharmaceu, 14 (9), 1870. doi: 10.3390/pharmaceutics14091870
Al Khafaji, AS, & Donovan, MD (2021). Pharmaceu, 13 (5), 761. doi: 10.3390/pharmaceutics 13050761
Alp, G & Aydogan, N (2020). Euro J Pharmaceu Biopharmaceu, 149, 45-57. doi: 10.1016/j.ejpb.2020.01.017
Al-sayadi, GMH et al. (2024). OpenNano, 17, 100206. doi: 10.1016/j.onano.2024.100206
Bakshi, V et al. (2022). Materials Today: Proceedings, 66, 2342-2357. doi: 10.1016/j.matpr.2022.06.329
Banerjee, R, & Purkayastha, P (2017). Soft Matter, 13 (33), 5506-5508. doi: 10.1039/C7SM01198A
Bertrand, N et al. (2017). Nat Commun, 8 (1), 777. doi: 10.1038/s41467-017-00600-w
Beule, AG (2010). GMS Cur Topics Otorhinolaryngol, Head Neck Surg, 9, Doc07. doi: 10.3205/cto000071
Bourganis, V et al. (2018). Euro J Pharmaceu Biopharmaceu, 128, 337-362. doi: 10.1016/j.ejpb.2018.05.009
Bunjes, H & Hunru, T (2007). Adv Drug Del Rev, 59 (6), 379-402. doi: 10.1016/j.addr.2007.04.013
Butnarasu, C et al. (2023). Pharmaceu, 15 (2). doi: 10.3390/pharmaceutics15020380
Chung, S et al. (2023). Epilep Behav Rep, 21, 100581. doi: 10.1016/j.ebr.2022.100581
Correia, A C et al. (2024). Internat J Pharmaceu, 664, 124631. doi: 10.1016/j.ijpharm.2024.124631
Costa, C P et al. (2021). Acta Pharmaceutica Sinica B, 11 (4), 925-940. doi: 10.1016/j.apsb.2021.02.012
de Oliveira Jr, E R et al. (2020). 10 (6), 1688-1699. doi: 10.1007/s13346-020-00816-2
England, R J A et al. (1999). Clin Otolaryngol Allied Sci, 24 (1), 67-68. doi: 10.1046/j.1365-2273.1999.00223.x
Ensign, L M et al. (2012). Adv Mat, 24 (28), 3887-3894. doi: 10.1002/adma.201201800
Fissan, H et al. (2014). Analytical Methods, 6 (18), 7324. doi: 10.1039/C4AY01203H
Food and Drug Administration (FDA). (2012). Retrieved from https://www.accessdata.fda.gov/drugsatfda_docs/label/2014/203093s000lbl.pdf
Gänger, S & Schindowski, K (2018). Pharmaceu, 10 (3), 116. doi: 10.3390/pharmaceutics 10030116
Gao, X et al. (2023). J Controlled Release, 353, 366-379. doi: 10.1016/j.jconrel.2022.11.051
Gao, Y et al. (2024). Bioeng Translatl Med, 9 (1). doi: 10.1002/btm2.10600
Ghasemiyeh, P & Mohammadi-Samani, S (2018). Res Pharmaceu Sci, 13 (4), 288. doi: 10.4103/1735-5362.235156
Gupta, S et al. (2017). BioMed Res Internat, 2017, 1-18. doi: 10.1155/2017/5984014
Hasan, N et al. (2021). Internat J Pharmaceu, 599, 120428. doi: 10.1016/j.ijpharm.2021.120428
Hua, T et al. (2024). Exp Opin Drug Del, 21 (4), 553-572. doi: 10.1080/17425247.2024.2339335
Huckaby, J T & Lai, SK (2018). Adv Drug Del Rev, 124, 125-139. doi: 10.1016/j.addr.2017.08.010
Kakad, S & Kshirsagar, S (2021). Heliyon, 7 (11), e08368. doi: 10.1016/j.heliyon.2021.e08368
Kakkar, D et al. (2015). Med Chem Comm, 6 (8), 1452-1463. doi: 10.1039/C5MD00104H
Kaplan, JE (2020 July 28). WebMD. Retrieved from https://www.webmd.com/hiv-aids/aids-hiv-medication
Killingsworth, L & Spudich, S (2022). Seminars Immunopath, 44 (5), 709-724. doi: 10.1007/s00281-022-00953-5
Knop, K et al. (2010). Angewandte Chemie International Edition, 49 (36), 6288-6308. doi: 10.1002/anie.200902672
Kurano, T et al. (2022). J Controlled Release, 344, 225-234. doi: 10.1016/j.jconrel.2022.03.017
Lai, S K et al. (2009). Adv Drug Del Rev, 61 (2), 158-171. doi: 10.1016/j.addr.2008.11.002
Lindl, K A et al. (2010). J Neuroim Pharmacol, 5 (3), 294-309. doi: 10.1007/s11481-010-9205-z
Li, Y et al. (2014). Biomat, 35 (30), 8467-8478. doi: 10.1016/j.biomaterials.2014.06.032
Maisel, K et al. (2016). Nanomed, 11 (11), 1337-1343. doi: 10.2217/nnm-2016-0047
Mehrotra, S et al. (2023). J Drug Del Sci Tech, 87, 104833. doi: 10.1016/j.jddst.2023.104833
Mistry, A et al. (2009). Internat J Pharmaceu, 379 (1), 146-157. doi: 10.1016/j.ijpharm.2009.06.019
Osborne, O et al. (2020). Trends Neurosci (Vol. 43, Issue 9). doi: 10.1016/j.tins.2020.06.007
Pelaz, B et al. (2015). ACS Nano, 9 (7), 6996-7008. doi: 10.1021/acsnano.5b01326
Prajapati, JB & Patel, GC (2021). J Drug Del Sci Tech, 63, 102377. doi: 10.1016/j.jddst.2021.102377
Rabanel, J-M et al. (2019). Nanoscale, 11 (2), 383-406. doi: 10.1039/C8NR04916E
Saini, S et al. (2021). Colloids and Surfaces B: Biointerfaces, 205, 111838. doi: 10.1016/j.colsurfb.2021.111838
Samaridou, E & Alonso, MJ (2018). Bioorg Med Chem, 26 (10), 2888-2905. doi: 10.1016/j.bmc.2017.11.001
Samaridou, E et al. (2020). Biomaterials, 230, 119657. doi: 10.1016/j.biomaterials.2019.119657
Sarma, A & Das, M (2020). Mol Biomed, 1 (1), 15. doi: 10.1186/s43556-020-00019-8
Saxena, S K et al. (2013). InTech. doi: 10.5772/55100
Schlachet, I & Sosnik, A (2019). ACS Applied Materials Interfaces, 11 (24), 21360-21371. doi: 10.1021/acsami.9b04766
Gilroy, SA (2023). Medscape.
Shi, L et al. (2021). Nanoscale, 13 (24), 10748-10764. doi: 10.1039/D1NR02065J Sibinovska, N et al. (2019). Euro J Pharmaceu Biopharm, 145, 85-95. doi: 10.1016/j.ejpb.2019.10.008
Suk, J S et al. (2016). Adv Drug Del Rev, 99, 28-51. doi: 10.1016/j.addr.2015.09.012
Tafech, B et al. (2024). Adv Healthcare Materials, 13 (18). doi: 10.1002/adhm.202304525
The Global HIV/AIDS Epidemic. (2021 November 30). HIV.Gov. Retrieved from https://www.hiv.gov/hiv-basics/overview/data-and-trends/global-statistics
U.S. Statistics of HIV. (2021 June 2). HIV.Gov. Retrieved from https://www.hiv.gov/hiv-basics/overview/data-and-trends/statistics
Verhoef, JJF & Anchordoquy, TJ (2013). Drug Del Translat Res, 3 (6), 499-503. doi: 10.1007/s13346-013-0176-5
Vijayan, V et al. (2017). Frontiers Immun, 8. doi: 10.3389/fimmu.2017.00580
Wang, Y-Y et al. (2008). Angewandte Chemie International Edition, 47 (50), 9726-9729. doi: 10.1002/anie.200803526
Wang, Y et al. (2020). Neurology, 95 (19), e2610-e2621. doi: 10.1212/WNL.0000000000010752
Waymack, JR & Sundareshan, V (2021). StatPearls. Retrieved from https://www.ncbi.nlm.nih.gov/books/NBK537293/
Williams, W et al. (2006). Am J Respir Cell Mol Bio, 34 (5), 527-536. doi: 10.1165/rcmb.2005-0436SF
Xu, Q et al. (2015). ACS Nano, 9 (9), 9217-9227. doi: 10.1021/acsnano.5b03876
Yasir, M et al. (2021). J Drug Delivery Sci Technol, 61, 102164. doi: 10.1016/j.jddst.2020.102164
Yasir, M & Sara, UVS (2014). Acta Pharmaceutica Sinica B, 4 (6), 454 463. doi: 10.1016/j.apsb.2014.10.005
Yuan, H et al. (2013). Mole Pharmaceu, 10 (5), 1865-1873. doi: 10.1021/mp300649z

We claim:

1. A nanocarrier enabled for delivery of at least one drug or therapeutic agent, comprising a solid lipid core and polyethylene glycol (PEG) chains attached to the surface of the core, wherein the at least one drug or therapeutic agent is encapsulated within the solid lipid core.

2. The nanocarrier of claim 1, wherein at least two drugs or therapeutic agents are encapsulated with the solid lipid core.

3. The nanocarrier of claim 2, wherein the at least two drugs or therapeutic agents are antiviral drugs.

4. The nanocarrier of claim 3, wherein the antiviral drugs are antiretroviral drugs.

5. The nanocarrier of claim 4, wherein the antiretroviral drugs are atazanavir and elvitegravir.

6. A method of preparing surface PEGylated solid lipid core nanocarriers comprising at least one drug or therapeutic agent, comprising the steps of
heating an oil phase solution comprising the at least one drug or therapeutic agent, polyethylene glycol, and a solvent to a selected temperature;
heating an aqueous solution comprising an emulsifier to the selected temperature
homogenizing the oil phase solution,
combining the aqueous solution with the oil phase solution during homeginization to produce an oil in water (O/W) emulsion,
sonicating the O/W emulsion to obtain an O/W nanoemulsion, and
cooling the O/W nanoemulsion to produce surface PEGylated solid lipid core nanocarriers which include the at least one drug or therapeutic agent in the solid lipid core.

7. The method of claim 6, wherein the at least one drug or therapeutic agent is an antiviral drug.

8. A method for treating AIDS in a subject in need thereof, comprising the steps of
preparing surface PEGylated solid core nanocarriers (SCNs) comprising at least two antiretroviral drugs, and
administering a therapeutically effective amount of the SCNs to the subject.

9. The method of claim 9, wherein the AIDS is neuroAIDS.

10. The method of claim 9, wherein the SCNs are administered intranasally.
